(19) United States
(12) Patent Application Publication
(10) Pub. No.: US 2024/0216446 A1
(43) Pub. Date: Jul. 4, 2024

(54) PROBIOTIC COMPOSITIONS USEFUL IN THE PREVENTION AND/OR TREATMENT OF METABOLIC SYNDROME AND RELATED DISORDERS

(71) Applicant: SYNBALANCE SRL, Origgio (VA) (IT)

(72) Inventors: Rosella MALANCHIN, Origgio (VA) (IT); Cristiana PIANGIOLINO, Origgio (VA) (IT); Silvia CASTEGNARO, Origgio (VA) (IT); Federica CARLOMAGNO, Origgio (VA) (IT); Chiara PESCIAROLI, Origgio (VA) (IT)

(21) Appl. No.: 18/288,361

(22) PCT Filed: May 9, 2022
(86) PCT No.: PCT/IB2022/054273

§ 371 Date: Oct. 25, 2023

(30) Foreign Application Priority Data
May 7, 2021 (IT) 102021000011774

(51) Int. Cl. A61K 35/747; A61K 35/745; A61P 3/00; A61P 3/04; A61P 3/06; A61P 3/10

(52) U.S. Cl. CPC A61K 35/747; A61K 35/745; A61P 3/00; A61P 3/04; A61P 3/06; A61P 3/10

(57) ABSTRACT

The present invention relates to compositions comprising a probiotic combination comprising two or more strains belonging to different species selected from: Lactobacillus acidophilus, Lactobacillus plantarum, Lactobacillus reuteri, Lactobacillus gasseri. Lactobacillus rhamnosus, Bifidobacterium lactis; useful in the prevention and/or treatment of metabolic syndrome and related disorders.

### TECHNICAL FIELD OF THE INVENTION

The present invention relates to compositions comprising a probiotic combination comprising two or more bacterial strains belonging to different species selected from: Lactobacillus acidophilus, Lactobacillus plantarum, Lactobacillus reuteri, Lactobacillus gasseri, Lactobacillus rhamnosus, Bifidobacterium lactis; useful in the prevention and/or treatment of metabolic syndrome and related disorders. In addition, the present invention refers to a probiotic combination, and a composition containing said combination, comprising two or more bacterial strains belonging to different species selected from a group comprising or, alternatively, consisting of: Lactobacillus gasseri and Bifidobacterium lactis, preferably said combination and said composition being for use in a method for the prevention and/or treatment of metabolic syndrome and related disorders.

### STATE OF THE ART

According to the World Health Organization, cardiovascular diseases are still the leading cause of death with an estimated 17.9 million every year. There are several risk factors linked to cardiovascular diseases, some of which are hereditary and not modifiable, while others are modifiable such as blood pressure, cholesterol and bad habits (diet, sedentary lifestyle, smoking, etc.). The biggest problem is that most people are not aware of their health status and this leads to a higher risk of developing cardiovascular disease.

To date, it's known how the combination of several risk factors can be an alarm bell for the development of cardiovascular disease. For example, hepatic steatosis has become the most common chronic liver disease in developed countries, representing a serious problem both clinically and economically. Similarly, metabolic syndrome, considered as a cluster of interconnected factors, greatly increases the risk of developing cardiovascular diseases. Metabolic syndrome refers a cluster of risk factors related to conditions that increase the chance of developing cardiovascular disease and diabetes. Subjects with metabolic syndrome have twice the risk of developing heart disease and five times the risk of developing diabetes. At worldwide level, it is estimated that 31% of the population is affected, reaching 55% in the elderly population.

The main risk factors associated with metabolic syndrome are different: the higher is the number of affecting conditions, the greater is the possibility of developing metabolic syndrome.

The most common conditions attributable to the onset of metabolic syndrome are:
High body fat concentration, especially at abdominal level, called visceral fat and high waist circumference.
High blood levels of LDL cholesterol and triglycerides and low blood levels of HDL;
Arterial hypertension;
Insulin resistance;
Hyperuricemia.

According to the World Health Organization (WHO) metabolic syndrome is confirmed if an altered blood sugar (or insulin resistance) and two of the following parameters:
central obesity, waist/hips ratio>0.9 in males; >0.85 in females and/or Body Mass Index (BMI)>30;
arterial pressure≥160/90 or antihypertensive drugs intake;
HDL cholesterol<35 mg/dl in males; <39 mg/dl in females;
Triglycerides≥ 150 mg/dl;
microalbuminuria>20 μg/min or albumin/creatinine ratio >20 mg/g (presence of traces of albumin in urine).
   occur at the same time.

It has been observed that a number of markers of systemic inflammation increases with the onset of metabolic syndrome, such as C-reactive protein, fibrinogen, interleukin 6, tumor necrosis factor alpha (TNF-α) and others. For example, C-reactive protein is produced by the liver and is found in peripheral blood. Its release into the bloodstream occurs in response to inflammatory processes and therefore its levels in the blood increase enormously if an inflammation is taking place. Together with TNF-α, it is considered an acute phase protein. In cardiology, the high-sensitivity C-reactive protein test allows to observe even the slightest changes in concentration, useful for monitoring cardiovascular risk. Along with other tests, such as cholesterol and triglycerides assessment, C-reactive protein dosage is a useful tool for determining risk factors associated with cardiovascular diseases.

To date, to prevent the onset of metabolic syndrome, it is necessary to improve people's modus vivendi in terms of good habits aimed at:
maintaining a healthy weight, avoiding conditions such as overweight or obesity with a special attention to visceral fat;
increasing physical activity;
following a balanced diet rich in fruits and vegetables.

To date, the treatments indicated for metabolic syndrome refer to a reduction of health risks linked to the above listed points.

According to the physician judgement, dedicated drugs can be prescribed depending on the risk factors involved.

In view of the above, there is still a need to identify alternative compositions effective in the prevention and/or treatment of metabolic syndrome and related disorders.

### SUMMARY OF THE INVENTION

The present invention relates to compositions comprising a probiotic combination comprising two or more strains belonging to different species selected from: Lactobacillus acidophilus, Lactobacillus plantarum (also known as: Lactiplantibacillus plantarum), Lactobacillus reuteri (also known as: Limosilactobacillus reuteri), Lactobacillus gasseri, Lactobacillus rhamnosus (also known as: Lacticaseibacillus rhamnosus), Bifidobacterium lactis, provided that when the probiotic combination consists of Lactobacillus acidophilus, Lactobacillus plantarum and Lactobacillus reuteri, said composition does not include inulin and fructo-oligosaccharides.

The invention further relates to said compositions for use as a medicament, particularly in the prevention and/or treatment of metabolic syndrome and related disorders.

In addition, the present invention refers to a probiotic combination, and a composition containing said combination, comprising two or more bacterial strains belonging to different species selected from the group comprising or, alternatively, consisting of: Lactobacillus gasseri and Bifidobacterium lactis, preferably said combination and said composition being for use in a method for the prevention and/or treatment of metabolic syndrome and related disorders.

### DETAILED DESCRIPTION OF THE INVENTION

The present invention relates to compositions comprising a probiotic combination comprising two or more strains belonging to different species selected from: Lactobacillus acidophilus, Lactobacillus plantarum (also known as Lactiplantibacillus plantarum), Lactobacillus reuteri (also known as: Limosilactobacillus reuteri), Lactobacillus gasseri, Lactobacillus rhamnosus (also known as: Lacticaseibacillus rhamnosus), Bifidobacterium lactis.

It has surprisingly been found that compositions comprising a probiotic combination comprising two or more strains belonging to different species selected from: Lactobacillus acidophilus, Lactobacillus plantarum, Lactobacillus reuteri, Lactobacillus gasseri, Lactobacillus rhamnosus, Bifidobacterium lactis, are effective in the prevention and/or treatment of metabolic syndrome and related disorders. It has also been found that a probiotic combination and a composition containing said probiotic combination, both comprising two or more bacterial strains belonging to different species selected from the group comprising or, alternatively, consisting of: Lactobacillus gasseri and Bifidobacterium lactis, are useful and effective for use in a method for the prevention and/or treatment of metabolic syndrome, in particular in the prevention and/or treatment of related disorders selected from the group comprising or, alternatively, consisting of: dyslipidemia, hypercholesterolemia, hypertriglyceridemia; overweight and obesity.

It is an object of the present invention a probiotic combination comprising two or more strains belonging to different species selected from: Lactobacillus gasseri and Bifidobacterium lactis, wherein said probiotic combination comprises or, alternatively, consists of the bacterial strain of Lactobacillus gasseri LG050 deposited at BCCM (Belgian Coordinated Collections of Micro-organisms)—LMG (Laboratorium voor Microbiologie-Bacteriënverzamelig) under the accession number “LMG P-29638” on 24 May 2016 and the bacterial strain of Bifidobacterium lactis BL050 deposited at DSMZ—DeutscheSammlung von Mikroorganismen und Zellkulturen GmbH under the accession number “DSM 25566” on 17 Jan. 2012.

Preferably, said probiotic combination comprising or, alternatively, consisting of a bacterial strain of Lactobacillus gasseri LG050 and a bacterial strain of Bifidobacterium lactis BL050 is used as a medicament; preferably it is for use in the prevention and/or treatment of metabolic syndrome, in particular said combination is for use in the prevention and/or treatment of related disorders selected from the group comprising or, alternatively, consisting of: dyslipidemias, hypercholesterolemia, hypertriglyceridemia; overweight and obesity.

In particular, it has been found that a probiotic combination (and a composition containing said probiotic combination) both comprising or, alternatively, consisting of a bacterial strain of Lactobacillus gasseri LG050 and a bacterial strain of Bifidobacterium lactis BL050 administered (for example, in an effective dose, one, two or three times a day depending on the amount of CFU of the strains) to a subject in need thereof for a period of time preferably from 2 weeks to 8 weeks, for example 3, 4, 5, 6 or 7 weeks, are able to affect the lipid profile of said subject with a reduction/decrease in total cholesterol, LDL and triglycerides values. A reduction in waist circumference and a significant reduction in the inflammatory marker C-reactive protein have been also observed.

Therefore, it is an object of the present invention a probiotic combination and a composition comprising said combination, both comprising or, alternatively, consisting of a bacterial strain of Lactobacillus gasseri LG050 and a bacterial strain of Bifidobacterium lactis BL050, for use in a method for the reduction/decrease of total cholesterol and/or LDL and/or triglycerides values, preferably the combination or composition is administered to a subject in need thereof in an effective dose, for example once a day, depending on the amount of CFU of the bacterial strains administered, for example for a period of 30 days.

Another object of the present invention is a composition comprising a probiotic combination comprising two or more strains belonging to different species selected from: Lactobacillus gasseri and Bifidobacterium lactis, wherein said probiotic combination comprises or, alternatively, consists of: the bacterial strain of Lactobacillus gasseri LG050 deposited at BCCM (Belgian Coordinated Collections of Micro-organisms)—LMG (Laboratorium voor Microbiologie-Bacteriënverzamelig) under the accession number “LMG P-29638” on 16 May 2024 and the bacterial strain of Bifidobacterium lactis BL050 deposited at DSMZ—DeutscheSammlung von Mikroorganismen und Zellkulturen GmbH under the accession number “DSM 25566” on 17 Jan. 2012 and, optionally, at least one physiologically acceptable excipient and/or vehicle.

Preferably, said at least one physiologically acceptable excipient and/or vehicle is selected from a group comprising or, alternatively, consisting of maltodextrin, inulin, fructo-oligosaccharides and/or acacia fibre.

Preferably, the composition comprising wherein said probiotic combination which comprises or, alternatively, consists of Lactobacillus gasseri LG050 and Bifidobacterium lactis BL050 and, optionally, at least one physiologically acceptable excipient and/or vehicle selected from the group comprising or, alternatively, consisting of maltodextrin, inulin, fructo-oligosaccharides and/or acacia fibre, is for use as a medicament. Preferably, said composition is for use in the prevention and/or treatment of metabolic syndrome, in particular said combination is for use in the prevention and/or treatment of disorders related to metabolic syndrome, selected from the group comprising or, alternatively, consisting of: dyslipidaemia, hypercholesterolemia, hypertriglyceridemia; overweight and obesity.

According to a preferred aspect of the invention, the compositions comprise a probiotic combination comprising Lactobacillus acidophilus, Lactobacillus plantarum, Lactobacillus reuteri provided that when the probiotic combination consists of Lactobacillus acidophilus, Lactobacillus plantarum and Lactobacillus reuteri, said composition does not include inulin and fruit-oligosaccharides, in particular the composition may comprise a probiotic combination comprising Lactobacillus acidophilus PBS066, Lactobacillus plantarum PBS067 and Lactobacillus reuteri PBS072.

According to another preferred aspect of the invention, the compositions comprise a probiotic combination comprising Lactobacillus gasseri and Bifidobacterium lactis, in particular the composition may comprise a probiotic combination comprising Lactobacillus gasseri LG050 and Bifidobacterium lactis BL050.

According to a further preferred aspect of the invention, the compositions comprise a probiotic combination comprising Lactobacillus rhamnosus and Lactobacillus reuteri, in particular the composition may comprise a probiotic combination comprising Lactobacillus rhamnosus LRH020 and Lactobacillus reuteri PBS072.

Lactobacillus acidophilus (L. acidophilus o LA) strain called “PBS066” was deposited at the DSMZ—Deutsche Sammlung von Mikroorganismen und Zellkulturen GmbH under the accession number “DSM 24936” on 17 Jun. 2011, according to the Budapest Treaty.

Lactobacillus plantarum (L. plantarum o LP) strain called “PBS067” was deposited at DSMZ—Deutsche Sammlung von Mikroorganismen und Zellkulturen GmbH under the accession number “DSM 24937” on 17 Jun. 2011, according to the Budapest Treaty.

Lactobacillus reuteri (L. reuteri o LR) strain called “PBS072” was deposited at DSMZ—DeutscheSammlung von Mikroorganismen und Zellkulturen GmbH under the accession number “DSM 25175” on 14 Sep. 2011, according to the Budapest Treaty. Lactobacillus gasseri (L. gasseri o LG) strain called “LG050” was deposit at BCCM (Belgian Coordinated Collections of Micro-organisms)—LMG (Laboratorium voor Microbiologie-Bacteriënverzamelig under the accession number “LMG P-29638” on 24 May 2016, according to the Budapest Treaty.

Bifidobacterium lactis (B. lactis o BL) strain called “BL050” was deposited at DSMZ—DeutscheSammlung von Mikroorganismen und Zellkulturen GmbH under the accession number “DSM 25566” on 17 Jan. 2012, according to the Budapest Treaty.

Lactobacillus rhamnosus (L. rhamnosus o LRH) strain called “LRH020” was deposited at DSMZ—Deutsche Sammlung von Mikroorganismen und Zellkulturen GmbH under the accession number “DSM 25568” on 17 Jan. 2012, according to the Budapest Treaty.

The present invention relates to compositions comprising a probiotic combination as above defined, as an active ingredient, and at least a physiologically acceptable excipient and/or vehicle.

Preferably, said at least one physiologically acceptable excipient and/or vehicle is selected from the group comprising or, alternatively, consisting of maltodextrin, inulin, fructo-oligosaccharides and/or acacia fibre.

The compositions may be formulated by conventional methods. Preferred forms of administration are solid formulations, such as hard capsules, single or coupled sachets, sticks, oro-soluble sticks, tablets, granulates, or liquid formulations, such as mono-dose or multi-dose bottles with dosing cap, multi-dose dispersions in oily phase, drops, syrups, multi-phase emulsions, etc.

Lactobacillus acidophilus (LA), preferably the Lactobacillus acidophilus PBS066 strain, may be present in the composition, as a percentage by weight based on the total weight of the probiotic combination, from 20% to 80%, preferably from 30% to 70%, even more preferably it is equal to 42%.

Lactobacillus acidophilus (LA), preferably the Lactobacillus acidophilus PBS066 strain, may be present in each individual unit dose in an amount ranging from 0.5 to 5 billion CFU, preferably in an amount ranging from 1 to 3 billion CFU, more preferably it is present in an amount equal to 2 billion CFU.

Lactobacillus plantarum (LP), preferably the Lactobacillus plantarum PBS067 strain, may be present in the composition, as a percentage by weight based on the total weight of the probiotic combination, from 5% to 40%, preferably from 10% to 30%, even more preferably it is equal to 17%.

Lactobacillus plantarum (LP), preferably the Lactobacillus plantarum PBS067 strain, may be present in each individual unit dose in an amount ranging from 0.5 to 5 billion CFU, preferably in an amount ranging from 1 to 3 billion CFU, more preferably it is present in an amount equal to 2 billion CFU.

Lactobacillus reuteri (LR), preferably the Lactobacillus reuteri PBS072 strain, may be present in the composition, as a percentage by weight based on the total weight of the probiotic combination, from 20% to 80%, preferably from 30% to 70%, even more preferably it is equal to 42% and 60%.

Lactobacillus reuteri (LR), preferably the Lactobacillus reuteri PBS072 strain, may be present in each individual unit dose in an amount ranging from 0.5 to 10 billion CFU, preferably in an amount ranging from 1 to 5 billion CFU, preferably in amounts ranging from 1 to 5 billion CFU, more preferably it is present in an amount equal to 2 billion CFU and 3 billion CFU.

Lactobacillus gasseri (LG), preferably the Lactobacillus gasseri LG050 strain, may be present in the composition, as a percentage by weight based on the total weight of the probiotic combination, from 20% to 80%, preferably from 30% to 70%, even more preferably it is equal to 60%.

Lactobacillus gasseri (LG), preferably the Lactobacillus gasseri LG050 may be present in each individual unit dose in an amount ranging from 0.5 to 5 billion CFU, preferably in an amount ranging from 1 to 3 billion CFU, more preferably it is present in an amount equal to 2 billion CFU.

Bifidobacterium lactis (BL), preferably the Bifidobacterium lactis BL050 strain, may be present in the composition, as a percentage by weight based on the total weight of the probiotic combination, from 20% to 80%, preferably from 30% to 70%, even more preferably it is equal to 40%. Bifidobacterium lactis (BL), preferably the Bifidobacterium lactis BL050 strain, may be present in each individual unit dose in an amount ranging from 0.5 to 5 billion CFU, preferably in an amount ranging from 1 to 3 billion CFU, more preferably it is present in an amount equal to 2 billion CFU.

Lactobacillus rhamnosus (LRH), preferably the Lactobacillus rhamnosus LRH020 strain, may be present in the composition, as a percentage by weight based on the total weight of the probiotic combination, from 20% to 80%, preferably from 30% to 70%, even more preferably it is equal to 40%.

Lactobacillus rhamnosus (LRH), preferably the Lactobacillus rhamnosus LRH020 strain, may be present in each individual unit dose in an amount ranging from 0.5 to 5 billion CFU, preferably in an amount ranging from 1 to 3 billion CFU, more preferably it is present in an amount equal to 2 billion CFU.

The species present in the probiotic combination, as reported in Example 1, may be present in a weight ratio of 2.5:2.5:1 or in a ratio of 1:1:1 when expressed in CFU.

The species present in the probiotic combination, as reported in Example 2, may be present in a weight ratio of 1.5:1 or in a ratio of 1:1 when expressed in CFU.

The species present in the probiotic combination, as reported in Example 3, may be present in a weight ratio of 1.5:1 or in a ratio of 1:1 when expressed in CFU.

The probiotic compositions of the invention can be administered orally.

A further object of the invention is the use of compositions comprising a probiotic combination comprising two or more strains belonging to different species selected from: Lactobacillus acidophilus, Lactobacillus plantarum, Lactobacillus reuteri, Lactobacillus gasseri, Lactobacillus rhamnosus, Bifidobacterium lactis, as a medicament and in particular in the prevention and/or treatment of metabolic syndrome and related disorders.

According to a preferred aspect of the invention, the compositions comprise a probiotic combination comprising Lactobacillus acidophilus, Lactobacillus plantarum, Lactobacillus reuteri provided that when the probiotic combination consists of Lactobacillus acidophilus, Lactobacillus plantarum and Lactobacillus reuteri, the composition does not include inulin and fruit-oligosaccharides, in particular the composition may comprise a probiotic combination comprising Lactobacillus acidophilus PBS066, Lactobacillus plantarum PBS067 and Lactobacillus reuteri PBS072 for use as a medicament and in particular in the prevention and/or treatment of metabolic syndrome and related disorders.

According to another preferred aspect of the invention, the compositions comprise a probiotic combination comprising Lactobacillus gasseri and Bifidobacterium lactis, in particular the composition may include a probiotic combination comprising Lactobacillus gasseri LG050 and Bifidobacterium lactis BL050 for use as a medicament, in particular in the prevention and/or treatment of metabolic syndrome and related disorders.

According to a further preferred aspect of the invention, the compositions comprise a probiotic combination comprising Lactobacillus rhamnosus and Lactobacillus reuteri, in particular the composition may comprise a probiotic combination comprising Lactobacillus rhamnosus LRH020 and Lactobacillus reuteri PBS072 for use as a medicament, in particular in the prevention and/or treatment of metabolic syndrome and related disorders.

According to a further aspect of the invention, the compositions of the invention are useful in the prevention and/or treatment of metabolic syndrome, in particular in the prevention and/or treatment of related disorders such as:
dyslipidaemia, in particular hypercholesterolemia, hypertriglyceridemia;
overweight and obesity;
hypertension;
insulin-resistance and impaired glucose tolerance.

The following examples further illustrate the invention.

FRn embodiments of this invention are reported below.
FR1. Composition comprising a probiotic combination comprising two or more strains belonging to different species selected from: Lactobacillus acidophilus, Lactobacillus plantarum, Lactobacillus reuteri, Lactobacillus gasseri, Lactobacillus rhamnosus and Bifidobacterium lactis provided that, when the probiotic combination consists of Lactobacillus acidophilus, Lactobacillus plantarum and Lactobacillus reuteri, the composition does not include inulin and fructo-oligosaccharides.
FR2. Composition according to FR1, wherein the probiotic combination comprises: Lactobacillus acidophilus, Lactobacillus plantarum, Lactobacillus reuteri.
FR3. Composition according to FR2 wherein the probiotic combination comprises: Lactobacillus acidophilus PBS066, Lactobacillus plantarum PBS067 and Lactobacillus reuteri PBS072.
FR4. Composition according to FR1, wherein the probiotic combination comprises: Lactobacillus gasseri and Bifidobacterium lactis.
FR5. Composition according to FR4, wherein the probiotic combination comprises: Lactobacillus gasseri LG050 and Bifidobacterium lactis BL050.
FR6. Composition according to FR1, wherein the probiotic combination comprises: Lactobacillus rhamnosus and Lactobacillus reuteri.
FR7. Composition according to FR6, wherein the probiotic combination comprises: Lactobacillus rhamnosus LRH020 and Lactobacillus reuteri PBS072.
FR8. Composition according to FR1-7 for use as a medicament.
FR9. Composition according to FR1-7 for use in the prevention and/or treatment of metabolic syndrome.
FR10. Composition according to FR1-7 for use in the prevention and/or treatment of disorders selected from: dyslipidemia, hypercholesterolemia, hypertriglyceridemia; overweight and obesity; hypertension; insulin resistance and impaired glucose tolerance.

### EXAMPLES

### Formulative Examples

### Example 1

A composition in oro-soluble stick form has been prepared, containing:

TABLE 1

| INGREDIENTS | % wt/wt | BILLION/DOSE |
|---|---|---|
| L. acidophilus PBS066 | 2.75 | 2 |
| L. plantarum PBS067 | 1.65 | 2 |
| L. reuteri PBS072 | 2.75 | 2 |
| Folic acid | 0.003 | — |
| Vitamin B12 | 0.035 | — |
| Vitamin B6 | 0.022 | — |
| Sorbitol | 69.4 | — |
| Silicon dioxide | 1 | — |
| Maltodextrin | 22.39 | — |

### Example 2

A composition in sachet form has been prepared, containing:

TABLE 2

| INGREDIENTS | % wt/wt | BILLION/DOSE |
|---|---|---|
| L. gasseri LG050 | 0.83 | 2 |
| B. lactis BL050 | 0.5 | 2 |
| Acacia fiber | 75 | — |
| Aroma | 1.88 | — |
| Silicon dioxide | 2 | — |
| Maltodextrin | 19.42 | — |

### Example 3

A composition in capsule form has been prepared, containing:

TABLE 3

| INGREDIENTS | % wt/wt | BILLION/DOSE |
|---|---|---|
| L. reuteri PBS072 | 21.43 | 3 |
| L. rhamnosus LRH020 | 14.29 | 3 |
| Magnesium stearate | 2 | — |
| Silicon dioxide | 2 | — |
| Maltodextrin | 2 | — |

### Experimental Examples

The following experimental examples are related to the evaluation of the efficacy of the compositions of the invention in the prevention and/or treatment of metabolic syndrome and related disorders.

### Example 4

A clinical study was carried out to determine the efficacy of the composition of Example 2 (L. gasseri LG050 and B. lactis BL050) in the prevention and/or treatment of disorders related to metabolic syndrome.

### Materials and Methods

For this study 30 healthy subjects (16 men and 14 women, average age 41±2) were enrolled, with suboptimal cholesterol values (average total cholesterol: 235.9±17.9), with a similar BMI (25.3±1.6), not receiving any drug therapy (with the exception of oral contraceptives), not affected by metabolic or gastrointestinal disorders and not taking other nutritional supplements during the trial. Before the start of the treatment, volunteers underwent to a period of standardization of eating habits for 14 days and were then instructed to take the probiotic composition reported in Example 2 for 30 days.

The study was divided into 3 checkpoints: before standardisation (T-14), at the start of the study (TO) and end of the study (T30). To assess the improvement of plasma cholesterol levels by the probiotic composition reported in Example 2, blood samples were taken at each check-point.

The observed parameters were: body mass index, waist circumference, blood pressure, total cholesterol, LDL-C, HDL-C, non HDL-C, C-reactive protein, etc.

Statistical analysis was carried out by comparing both the standardization period (T-14) with the start of treatment and the end of treatment (T30) versus the start (TO). A t-test was carried out on the coupled samples, which have been considered significant with a p-value <0.05.

### Results

The values obtained, expressed as mean value with standard deviation, during the three checkpoints are reported in Table 4.

TABLE 4

| | T −14 | T0 | T30 |
|---|---|---|---|
| Age (years) | 41 ± 2 | | |
| Body Mass Index (Kg/m²) | 25.3 ± 1.6 | 25.2 ± 1.5 | 25.0 ± 1.4 |
| Waist circumference (cm) | 92 ± 3 | 91 ± 2 | 88 ± 3* |
| ICO | 0.52 ± 0.06 | 0.51 ± 0.05 | 0.50 ± 0.04 |
| Systolic pressure (mmHg) | 132.1 ± 6.2 | 132.0 ± 5.8 | 131.3 ± 5.7 |
| Diastolic pressure (mmHg) | 84.3 ± 2.2 | 85.1 ± 2.5 | 84.2 ± 2.2 |
| Arterial pressure (mmHg) | 48.4 ± 1.7 | 47.8 ± 1.7 | 47.1 ± 1.5 |
| Heart rate (bpm) | 71.0 ± 3.1 | 70.4 ± 3.8 | 69.8 ± 3.6 |
| Fasting plasma glucose (mg/dL) | 90.8 ± 4.9 | 92.1 ± 3.7 | 88.9 ± 3.9 |
| Total cholesterol (mg/dL) | 235.9 ± 17.9 | 222.8 ± 15.8° | 208.6 ± 15.4* |
| LDL-C (mg/dL) | 151.1 ± 12.1 | 143.2 ± 14.1° | 131.5 ± 11.9* |
| HDL-C (mg/dL) | 43.5 ± 2.3 | 44.9 ± 2.6 | 44.4 ± 2.9 |
| Non HDL-C (mg/dL) | 193.5 ± 15.2 | 178.4 ± 15.1° | 161.2 ± 13.0* |
| Triglycerides (mg/dL) | 215.1 ± 22.5* | 200.9 ± 21.3° | 199.4 ± 19.6 |
| Aspartate transaminase (U/L) | 23.5 ± 2.6 | 23.1 ± 3.3 | 22.5 ± 3.4 |
| Alanine transaminase (U/L) | 24.5 ± 2.3 | 24.4 ± 2.6 | 23.8 ± 2.7 |
| Gamma-glutamyl transferase (U/L) | 26.6 ± 4.4 | 26.3 ± 4.2 | 26.1 ± 4.1 |
| Serum uric acid (mg/dL) | 5.6 ± 0.7 | 5.7 ± 0.6 | 5.6 ± 0.6 |
| Creatinine (mg/dL) | 0.9 ± 0.2 | 0.9 ± 0.3 | 0.9 ± 0.3 |
| eGFR (ml/min) | 98.4 ± 5.0 | 97.9 ± 4.9 | 97.8 ± 4.1 |
| hsCRP (mg/L) | 2.76 ± 0.21 | 2.80 ± 0.24 | 2.61 ± 0.19* |

*p < 0.05 vs. baseline;
°p < 0.05 vs. pre-diet
eGFR = Estimated glomerular filtration rate; GGT = Gamma-glutamyl transferase; HDL = High-density lipoprotein; HOMA-IR = Homeostatic model assessment for insulin resistance; hsCRP = high sensitivity C-reactive protein; ICO = Index of central obesity; LDL = Low-density lipoprotein.

The results reported in Table 4 show how the intake for a limited period of 30 days can affect the lipid profile with a decrease in total cholesterol, LDL and triglycerides. A reduction in waist circumference and a significant reduction in the inflammatory marker C-reactive protein were also observed.

### Example 5

In-vitro studies have been carried out to determine the ability of the strains of the invention to modulate the absorption of cholesterol present in the culture broth. Each culture medium was enriched with 100 ug/ml of cholesterol and 0.3% of oxidized bile, then 1% of individual strains were added and incubated anaerobically for 20 hours at 37° C.

The residual cholesterol was measured by spectrophotometer. The cholesterol absorbed by the probiotic strains was calculated as the difference in cholesterol measured before probiotic inoculation and after inoculation, expressed as ug/mL. An increase in absorbed cholesterol is indicative of the fact that the probiotics are able to remove it from the system and use it for their metabolism, potentially reducing its bioavailability to the host system. The results are shown in Table 5.

TABLE 5

| | Cholesterol added | Cholesterol measured | Absorbed |
|---|---|---|---|
| | in the system | after incubation | cholesterol |
| LA - PBS066 | 100 | 56 | 44 |
| LG - LG050 | 99.2 | 23.6 | 75.6 |
| LR - PBS072 | 99.6 | 36.8 | 62.8 |
| LRh - LRH020 | 99.3 | 46 | 53.2 |
| BL - BL050 | 100.1 | 73.3 | 26.7 |
| LP - PBS067 | 99.2 | 37.2 | 61.9 |

### Example 6

In-vitro studies have been carried out to determine the ability of the strains of the invention to modulate the absorption of cholesterol from the culture broth of each probiotic strain enriched with 100 ug/ml of cholesterol alone and in the presence of bile salts such as shown in Table 6.

TABLE 6

| | |
|---|---|
| Culture | Culture medium enriched with 2.8 Mm of glycocholate and |
| medium A | 1.2 Mm of taurocholate and 100 ug/ml of cholesterol |
| Culture | Culture medium enriched with 100 ug/ml of cholesterol |
| medium B | |

The single strains have been added at 1% to the culture medium and incubated anaerobically at 37° C. for 20 hours. Residual cholesterol has been measured by spectrophotometer. Co-precipitated cholesterol was calculated as the difference in the results of residual cholesterol in the presence and absence of bile salts (difference in cholesterol measured in medium A and medium B after incubation). Co-precipitation of cholesterol affects the reduction of absorbable cholesterol. The results are given as mean values expressed in ug/ml in Table 7.

TABLE 7

| | Cholesterol | Cholesterol | |
|---|---|---|---|
| | measured in | measured in | |
| | culture medium | culture medium | Co-precipi- |
| | B after | A after | tated |
| | incubation | incubation | Cholesterol |
| LA - PBS066 | 47.7 | 53.7 | 6 |
| LG - LG050 | 23.0 | 25.8 | 2.8 |
| LR - PBS072 | 33.8 | 36.1 | 2.3 |
| LRh - LRH020 | 44.7 | 48.0 | 3.3 |
| BL - BL050 | 71.1 | 75.2 | 4.1 |
| LP - PBS067 | 40.2 | 42.2 | 2.1 |

### Example 7

In-vitro studies have been carried out to determine the intrinsic activity of the strains of the invention on the enzyme involved in the de-conjugation of bile salts (Bile Salt hydrolase).

The strains were assessed for their ability to de-conjugate the following bile salts:
Glycocholate,
Glycodeoxycholate,
Taurocholate,
Taurodeoxycholate.

The strains were incubated with the different substrates for 24 hours. Subsequently the enzymatic activity was evaluated by measuring the amount of free amino acids through derivatization with ninhydrin. An increase in the concentration of free amino acids indicates a higher enzymatic activity and consequently a greater de-conjugation of bile salts, which can be attributed to a reduction in the absorption of cholesterol. The results are reported as mean and expressed in U/mL with the relative standard deviation. The results are reported in Tables 8, 9, 10 and 11.

TABLE 8

| Glycocholate | Mean | Standard Deviation |
|---|---|---|
| LP - PBS067 | 539.3 | 20.5 |
| LA - PBS066 | 235.7 | 17.4 |
| LR - PBS072 | 228.7 | 21.9 |
| LRh - LRH020 | 372.3 | 40.6 |
| BL - BL050 | 442.3 | 38.2 |
| LG - LG050 | 171.7 | 11.0 |

TABLE 9

| Glycodeoxycholate | Mean | Standard Deviation |
|---|---|---|
| LP - PBS067 | 337.0 | 32.7 |
| LA - PBS066 | 161.3 | 14.5 |
| LR - PBS072 | 114.7 | 3.1 |
| LRh - LRH020 | 239.3 | 13.3 |
| BL - BL050 | 292.3 | 4.7 |
| LG - LG050 | 149.3 | 2.5 |

TABLE 10

| Taurocholate | Mean | Standard Deviation |
|---|---|---|
| LP - PBS067 | 226.3 | 25.7 |
| LA - PBS066 | 138.7 | 16.2 |
| LR - PBS072 | 192.3 | 7.2 |
| LRh - LRH020 | 316.3 | 9.6 |
| BL - BL050 | 334.7 | 17.2 |
| LG - LG050 | 121.7 | 3.2 |

TABLE 11

| Taurodeoxycholate | Mean | Standard Deviation |
|---|---|---|
| LP - PBS067 | 218.7 | 6.4 |
| LA - PBS066 | 158.0 | 8.7 |
| LR - PBS072 | 194.3 | 5.8 |
| LRh - LRH020 | 246.7 | 9.6 |
| BL - BL050 | 338.3 | 11.2 |
| LG - LG050 | 131.3 | 7.2 |

### Example 8

In vitro studies have been carried out to determine the ability of the strains of the invention to modulate pancreatic lipase involved in lipid metabolism. The study was carried out on Balb 3T3 fibroblasts cell line.

The increase in enzymatic activity was measured by evaluating the synthesis of fatty acids from triglycerides, present as substrate in the reaction mix. An increase in activity is indicative of a greater use of lipid reserves. Enzyme activity was measured through a colorimetric assay after 24-hour and 5-day. The results, expressed as mean and as a percentage variation with respect to the control, are given in Table 12 and expressed in mU.

TABLE 12

| | | Lipase Activity (mU) | | |
|---|---|---|---|---|
| | 24 hours | Var % | 5 days | Var % |
| CTR | 50 | | 50 | |
| LR - PBS072 | 61.2 | 22.4% | 59.1 | 18.2% |
| LP - PBS067 | 58.8 | 17.6% | 58.1 | 16.2% |
| LA - PBS066 | 56.7 | 13.5% | 56.6 | 13.2% |
| LRh - LRH020 | 58.1 | 16.3% | 58.3 | 16.5% |
| LG - LG050 | 57.6 | 15.2% | 56.1 | 12.3% |

### Example 9

In vitro studies have been carried out to determine the ability of the strains of the invention to modulate the activity of the enzyme HMG-COA reductase involved in cholesterol biosynthesis. The study was carried out on Balb 3T3 fibroblasts cell line. The increase in enzymatic activity was measured by evaluating the synthesis of mevalonic acid, the precursor of the synthesis of sterols and isoprenoids, present as substrate in the reaction mix. The reduction of the activity is indicative of a non-hormonal control modulation of blood cholesterol levels. Enzyme activity was measured through a colorimetric assay after 24-hour and 5-day. The results, expressed as mean (expressed in mU) and as percentage variation with respect to the control, are reported in Table 13.

TABLE 13

| | Enzymatic activity of HMG-CoA reductase (mU) | | | |
|---|---|---|---|---|
| | 24 hour | Var % | 5 days | Var % |
| CTR | 100 | | 100 | |
| LR - PBS072 | 93.1 | −6.9% | 93.6 | −6.4% |
| LP - PBS067 | 94.6 | −5.4% | 91.6 | −8.4% |
| LA - PBS066 | 97.8 | −2.2% | 95.9 | −4.1% |
| LRh - LRH020 | 95.5 | −4.5% | 93.8 | −6.2% |
| LG - LG050 | 96.4 | −3.6% | 97.6 | −2.4% |

### Example 10

In vitro studies have been carried out to determine the ability of the strains of the invention to modulate the enzymes involved in carbohydrate metabolism (alpha-amylase, alpha-glucosidase and beta-glucosidase). The study was carried out on Balb 3T3 fibroblasts cell line. The decrease of the enzymatic activity is indicative of a non-hormonal control of blood sugar levels. Reduction of enzymatic activity was measured by evaluating the conversion of different substrates for the enzymes being tested:
starch for alpha-amylase,
saccharose for alpha-glucosidase,
lactose for beta-glucosidase.

Alpha-amylase activity was evaluated using Lugol reagent, while Fehling assay was used to evaluate alpha- and beta-glucosidase activity.

The results, expressed as mean (expressed in mU), percentage of digestion and percentage variation with respect to the control, are given in Tables 14, 15 and 16.

TABLE 14
α -amilase Activity

| | 24 hours | | | 5 days | | |
|---|---|---|---|---|---|---|
| | Digested | | | Digested | | |
| | starch | % | % vs | starch | % | % vs |
| | (mg) | digestion | CTR | (mg) | digestion | CTR |
| CTR | 86.5 | 57.7% | | 90.30 | 60.2% | |
| LR - PBS072 | 93 | 62% | 4.33% | 78.20 | 52.1% | −8.07% |
| LP - PBS067 | 91.6 | 61.1% | 3.40 | 85.50 | 57% | −3.20% |
| LA - PBS066 | 92.2 | 61.5% | 3.80% | 78.60 | 52.4% | −7.80% |
| LRh - RH020 | 90.40 | 60.3 | 2.60% | 77.40 | 51.6 | −8.60% |
| LG - LG050 | 91.6 | 61.2% | 3.4% | 83.1 | 55.4% | −4.80% |

TABLE 15
α - glucosidase Activity

| | 24 hours | | | | | |
|---|---|---|---|---|---|---|
| | Digested | | | Digested | 5 days | |
| | Saccharose | % | % vs | Saccharose | % | % vs |
| | (mg) | digestion | CTR | (mg) | digestion | CTR |
| CTR | 52.9 | 35.3% | | 59.60 | 39.7% | |
| LR - PBS072 | 56 | 37.3% | 2.07% | 47 | 31.3% | −8.40% |
| LP - PBS067 | 58.7 | 39.1% | 3.87% | 56.10 | 37.4% | −2.33% |
| LA- PBS066 | 57.50 | 38.3% | 3.07% | 51.8 | 34.5% | −5.20% |
| LRh-LRH020 | 58.10 | 38.7% | 3.47% | 52.4 | 34.9% | −4.80% |
| LG - LG050 | 56.20 | 37.5% | 2.20% | 53.3 | 35.5% | −4.2% |

TABLE 16
β - glucosidase activity

| | 24 hours | | | | | |
|---|---|---|---|---|---|---|
| | Digested | | | Digested | 5 days | |
| | Lactose | % | % vs | Lactose | % | % vs |
| | (mg) | digestion | CTR | (mg) | digestion | CTR |
| CTR | 62.20 | 41.5% | | 60.3 | 40.2% | |
| LR - PBS072 | 64.9 | 43.3% | 1.80% | 51.9 | 34.6% | −5.6% |
| LP - PBS067 | 67.60 | 45.1% | 3.60% | 57 | 38% | −2.2% |
| LA - PBS066 | 67.10 | 44.7% | 3.27% | 53.9 | 35.9% | −4.27% |
| LRh - LRH020 | 65.50 | 43.7% | 2.20% | 54.50 | 36.9% | −3.87% |
| LG - LG050 | 67.3 | 44.9% | 3.4% | 58.50 | 39% | −1.20% |

### Example 11

Ex-vivo studies have been carried out to determine the ability of the strains of the invention to modulate triglyceride turnover. In particular, the levels of metabolized triglycerides and the bioavailability of glycerol, a product resulting from the metabolism of triglycerides, were observed. It has been used an intestinal epithelium, grown on a support that allows exchanges between different departments, with the aim of mimicking what occurs at the level of the intestinal lumen and what is instead absorbed at the systemic level. An increase in the turnover of triglycerides is indicative of a use of its components both by probiotics for their metabolism and at the cellular level. A low percentage of glycerol bioavailability (resulting from the metabolism of triglycerides) is indicative of how much, at the end of the metabolic process, it is absorbed at the systemic level. The results, shown in Table 17, are expressed as a percentage.

TABLE 17

| | Metabolized | Bioavailable |
|---|---|---|
| | Triglycerides | glycerol |
| CTR- | 30% | 14% |
| LA - PBS066 | 42% | 5% |
| LP - PBS067 | 41% | 5% |
| LR - PBS072 | 69% | 5% |
| BL - BL050 | 37% | 4% |
| LG - LG050 | 35% | 5% |
| LRh - LRH020 | 21% | 7% |

### Example 12

Ex-vivo studies have been carried out to determine the ability of the strains of the invention to modulate the expression of the hormone associated with a reduction in the sense of hunger, peptide YY. An Increase in the concentration of this peptide is potentialy linked to a reduction in the sense of hunger and therefore of appetite. The results are given in Table 18 as an average and expressed in pp/mL.

TABLE 18

| | Peptide YY dosage |
|---|---|
| CTR- | 14.96 |
| LA - PBS066 | 32.46 |
| LP - PBS067 | 28.41 |
| LR - PBS072 | 22.38 |
| BL - BL050 | 23.63 |
| LG - LG050 | 20.84 |
| LRh - LRH020 | 42.82 |

1. A probiotic combination comprising two or more strains belonging to different species selected from: Lactobacillus gasseri and Bifidobacterium lactis, wherein said probiotic combination comprises the bacterial strain of Lactobacillus gasseri LG050 deposited at BCCM (Belgian Coordinated Collections of Micro-organisms)—LMG (Laboratorium voor Microbiologie—Bacteriënverzamelig) under the accession number “LMG P-29638” on 24 May 2016 and the bacterial strain of Bifidobacterium lactis BL050 deposited at DSMZ—DeutscheSammlung von Mikroorganismen und Zellkulturen GmbH under the accession number “DSM 25566” on 17 Jan. 2012.

2. The probiotic combination according to claim 1, wherein said combination consists of the bacterial strain of Lactobacillus gasseri LG050 and the bacterial strain of Bifidobacterium lactis BL050.

3. The combination according to claim 1, wherein said combination is for use as a medicament.

4. The combination according to claim 3, wherein said combination is for use in the prevention and/or treatment of metabolic syndrome.

5. The combination according to claim 4, wherein said combination is for use in the prevention and/or treatment of disorders related to metabolic syndrome, selected from the group comprising or, alternatively, consisting of:
dyslipidaemia, hypercholesterolemia, hypertriglyceridemia;
overweight and obesity.

6. A composition comprising a probiotic combination comprising two or more strains belonging to different species selected from: Lactobacillus gasseri and Bifidobacterium lactis, wherein said probiotic combination comprises the bacterial strain of Lactobacillus gasseri LG050 deposited at BCCM (Belgian Coordinated Collections of Micro-organisms)—LMG (Laboratorium voor Microbiologie—Bacteriënverzamelig) under the accession number “LMG P-29638” on 24 May 2016 and the bacterial strain of Bifidobacterium lactis BL050 deposited at DSMZ—DeutscheSammlung von Mikroorganismen und Zellkulturen GmbH under the accession number “DSM 25566” on 17 Jan. 2012 and, optionally, at least one physiologically acceptable excipient and/or vehicle.

7. The composition according to claim 6, wherein at least one physiologically acceptable excipient and/or vehicle has been selected from the group comprising or, alternatively, consisting of maltodextrin, inulin, fructo-oligosaccharides and/or acacia fibre.

8. The composition according to claim 6, wherein said combination consists of the bacterial strain of Lactobacillus gasseri LG050 and the bacterial strain of Bifidobacterium lactis BL050 and, optionally, at least one physiologically acceptable excipient and/or vehicle selected from the group comprising or, alternatively, consisting of maltodextrin, inulin, fructo-oligosaccharides and/or acacia fibre.

9. The composition according to claim 6, wherein said composition is for use as a medicament.

10. The composition according to claim 9, wherein said composition is for use in the prevention and/or treatment of metabolic syndrome.

11. The composition according to claim 10, wherein said composition is for use in the prevention and/or treatment of disorders related to metabolic syndrome, selected from a group comprising or, alternatively, consisting of dyslipidaemia, hypercholesterolemia, hypertriglyceridemia; overweight and obesity.

12. A method of treating a metabolic syndrome comprising: administering to a subject in need thereof a probiotic combination comprising two or more strains belonging to different species selected from: Lactobacillus gasseri and Bifidobacterium lactis, wherein said probiotic combination comprises the bacterial strain of Lactobacillus gasseri LG050 deposited at BCCM (Belgian Coordinated Collections of Micro-organisms)—LMG (Laboratorium voor Microbiologie-Bacteriënverzamelig) under the accession number “LMG P-29638” on 24 May 2016 and the bacterial strain of Bifidobacterium lactis BL050 deposited at DSMZ—DeutscheSammlung von Mikroorganismen und Zellkulturen GmbH under the accession number “DSM 25566” on 17 Jan. 2012.

13. The method of claim 12, herein the patient has dyslipidemia, hypercholesterolemia, hypertriglyceridemia, overweightness and/or obesity.

14. The method of claim 12, wherein the combination is administered in a physiologically acceptable excipient that comprises maltodextrin, inulin, fructo-oligosaccharides and/or acacia fibre.
